(19) United States
(12) Patent Application Publication
(10) Pub. No.: US 2024/0366373 A1
(43) Pub. Date: Nov. 7, 2024

(54) RELEASABLE RETAINING MECHANISMS FOR AN ACTUATOR ASSEMBLY OF A DELIVERY APPARATUS FOR AN EXPANDABLE PROSTHETIC DEVICE

(71) Applicant: EDWARDS LIFESCIENCES CORPORATION, Irvine, CA (US)

(72) Inventors: Elazar Levi Schwarcz, Netanya (IL); Eran Grosu, Kfar-Saba (IL)

(21) Appl. No.: 18/775,700

(22) Filed: Jul. 17, 2024

Related U.S. Application Data
(63) Continuation of application No. PCT/US2022/053692, filed on Dec. 21, 2022.
(60) Provisional application No. 63/302,960, filed on Jan. 25, 2022.

(51) Int. Cl. A61F 2/24

(52) U.S. Cl. CPC A61F 2/243; A61F 2/2418; A61F 2/2436

(57) ABSTRACT

Releasable retaining mechanisms for an actuator assembly of a delivery apparatus for an expandable prosthetic heart valve are disclosed. As one example, an actuator assembly can include an outer sleeve comprising a tube portion and an extension that extends distally from the tube portion and includes a first aperture in its distal end, an actuation member extending through the tube portion of the outer sleeve, and a retaining element configured to extend through the first aperture and hold the outer sleeve against an end portion of a frame of a prosthetic device. The retaining element is biased into a first position that holds the outer sleeve in engagement with the frame and is configured to flex in response to a pulling force into a second position that enables the retaining element to be released from the first aperture to disengage the outer sleeve from the end portion of the frame.

### CROSS-REFERENCE TO RELATED APPLICATIONS

This application is a continuation of PCT Application No. PCT/US2022/053692, filed Dec. 21, 2022, which claims the benefit of U.S. Provisional Patent Application No. 63/302,960, filed Jan. 25, 2022, both of which applications are incorporated by reference herein in their entireties.

### FIELD

The present disclosure relates to implantable, mechanically expandable prosthetic devices, such as prosthetic heart valves, and to delivery assemblies for, and including, such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Prosthetic heart valves that rely on a mechanical actuator for expansion can be referred to as “mechanically expandable” prosthetic heart valves. In some examples, an actuation assembly for a delivery apparatus for a mechanically expandable prosthetic heart valve can include a driver covered by an outer sleeve. The driver is configured to interface with and rotate a portion of a frame of the prosthetic heart valve while the outer sleeve is pressed against another portion of the frame to provide a counter-force during rotation of the driver, thereby causing radial expansion of the frame of the prosthetic heart valve.

### SUMMARY

Described herein are prosthetic heart valves, delivery apparatus, and methods for implanting prosthetic heart valves. The disclosed prosthetic heart valves, delivery apparatuses, and methods can, for example, provide mechanisms for releasably securing a sleeve of an actuator assembly of a delivery apparatus to a prosthetic heart valve. For example, a retaining element can be configured to secure an outer sleeve of an actuator assembly of the delivery apparatus to a frame of the prosthetic heart valve, during radial expansion of the frame via the actuator mechanism, and then be removed such that the actuator mechanism can be uncoupled from the frame and removed from the implantation site. As such, the devices and methods disclosed herein can, among other things, overcome one or more of the deficiencies of typical prosthetic heart valves and their delivery apparatus.

A delivery apparatus for a prosthetic device can comprise a handle and one or more shafts coupled to the handle.

In some examples, the delivery apparatus can comprise an actuator assembly, the actuator assembly comprising an outer sleeve with an extension, an actuation member extending through the outer sleeve, and a retaining element configured to extend through an aperture in the extension of the outer sleeve and hold the outer sleeve against an end portion of a frame of a prosthetic device.

In some examples, the retaining element is configured to hold the outer sleeve in engagement with the frame, and is configured to flex in response to a pulling force such that it is released from the aperture, thereby enabling disengagement of the outer sleeve from the end portion of the frame of the prosthetic device.

In some examples, the retaining element is biased into a first position where it holds the outer sleeve in engagement with the frame, and is configured to flex in response to a pulling force into a second position that enables the retaining element to be released from the first aperture, thereby enabling disengagement of the outer sleeve from the end portion of the frame of the prosthetic device.

In some examples, the actuator assembly extends distally from the handle.

In some examples, a flexible element extends from the handle to an end portion of the retaining element and the flexible element is configured to apply the pulling force to the end portion of the retaining element in response to actuation of a control mechanism of the handle.

In some examples, the retaining element comprises a head portion including a second aperture and two legs spaced apart from one another and extending from the head portion, wherein the two legs are configured to flex between the first position and the second position.

In some examples, the extension of the outer sleeve is a first extension, the outer sleeve comprises a second extension extending from an opposite side of the tube portion of the outer sleeve than the first extension, the first and second extensions spaced apart from one another, and each of the first extension and the second extension include two apertures therein that are spaced apart from one another in a direction that is perpendicular to an axial direction of the actuator assembly.

In some examples, the retaining element is a flexible tubular retaining element, and in the first position the retaining element assumes a U-shaped configuration with a first leg of the retaining element extending through a first aperture of the two apertures of the first extension and a first aperture of the two apertures of the second extension, the first apertures of the first and second extensions opposing one another across a gap separating the first and second extensions, and a second leg extending through a second aperture of the two apertures in the first extension and a second apertures in the two apertures in the second extension, the second apertures opposing one another across the gap separating the first and second extensions.

In some examples, a delivery apparatus for a radially expandable prosthetic device comprises an actuator assembly, the actuator assembly comprising an outer sleeve comprising a tube portion and an extension that extends distally from the tube portion, the extension comprising a first aperture in its distal end, an actuation member extending through the tube portion of the outer sleeve, and a retaining element configured to extend through the first aperture in the extension of the outer sleeve and hold the outer sleeve against an end portion of a frame of a prosthetic device. The retaining element is biased into a first position where it holds the outer sleeve in engagement with the frame, and is configured to flex in response to a pulling force into a second position that enables the retaining element to be released from the first aperture, thereby enabling disengagement of the outer sleeve from the end portion of the frame of the prosthetic device.

In some examples, a delivery apparatus comprises one or more of the components recited in Examples 1-15 below.

An assembly can comprise a prosthetic heart valve and a delivery apparatus.

In some examples, an assembly can comprise a prosthetic heart valve comprising a frame and a delivery apparatus comprising a handle and at least one actuator assembly extending from the handle. The actuator assembly comprises an outer sleeve having a tube portion abutting an outflow end portion of a frame member of the frame and a first extension that extends distally from the tube portion and over the frame member. The actuator assembly further comprises a retaining element extending through a first aperture in the first extension of the outer sleeve and a second aperture in the frame member, the retaining element configured to hold the outer sleeve in engagement with the frame member. The retaining element is configured to flex and release from the first and second apertures in response to a pulling force applied to an end portion of the retaining element.

In some examples, an assembly comprises a prosthetic heart valve, the prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another. The frame further comprises a threaded rod extending through the first and second inner bores. The assembly further comprises a delivery apparatus comprising: a handle and at least one actuator assembly extending from the handle, the actuator assembly comprising: an outer sleeve having a tube portion abutting an outflow end portion of the second frame member and a first extension that extends distally from the tube portion and over the second frame member, a driver extending through the tube portion of the outer sleeve and engaging an end of the threaded rod, the driver configured to rotate the threaded rod to radially expand and/or compress the frame, and a retaining element extending through a first aperture in the first extension of the outer sleeve and a second aperture in the second frame member, the retaining element configured to hold the outer sleeve in engagement with the second frame member. The retaining element is configured to flex and release from the first and second apertures in response to a pulling force applied to an end portion of the retaining element.

In some examples, an assembly comprises a prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another. The frame further comprises a threaded rod extending through the first and second inner bores. The assembly further comprises a delivery apparatus comprising: a handle, at least one actuator assembly extending from the handle, the actuator assembly comprising: an outer sleeve having a tube portion abutting an outflow end portion of the second frame member and a first extension that extends distally from the tube portion and over the second frame member, the first extension including a first aperture disposed in a distal end of the first extension; a driver extending through the tube portion of the outer sleeve and engaging an end of the threaded rod, the driver configured to rotate the threaded rod to radially expand the frame; and a retaining element releasably coupling the outer sleeve to the second frame member, the retaining element comprising a head portion and two legs extending distally from the head portion, wherein the two legs extend through the first aperture in the first extension and a second aperture in the second frame member and retain the outer sleeve in engagement with the second frame member.

In some examples, an assembly comprises a prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another, and a threaded rod extending through the first and second inner bores. The assembly further comprises a delivery apparatus comprising a handle and at least one actuator assembly extending from the handle, the actuator assembly comprising: an outer sleeve having a tube portion abutting an outflow end portion of the second frame member, first and second extensions that each extend distally from the tube portion and over the second frame member, the first and second extensions including two sets of aligned apertures. The actuator assembly further comprises a driver extending through the tube portion of the outer sleeve and engaging an end of the threaded rod, the driver configured to rotate the threaded rod to radially expand the frame, and a flexible elongate retaining element releasably coupling the outer sleeve to the second frame member, the retaining element assuming a u-shape comprising two legs when the retaining element is coupled to the outer sleeve and the second frame member. A first leg of the two legs extends through a first set of aligned apertures of the two sets of aligned apertures and a second leg of the two legs extends through a second set of aligned apertures of the two sets of aligned apertures such that the outer sleeve is retained in engagement with the second frame member.

In some examples, an assembly comprises one or more of the components recited in Examples 16-49 below.

A method can comprise advancing a distal end portion of a delivery apparatus toward a native heart valve, the delivery apparatus releasably coupled to a prosthetic heart valve by at least one actuator assembly, the prosthetic heart valve retained by the delivery apparatuses in a radially compressed state and comprising a frame comprising at least one actuation mechanism. The method further comprises radially expanding the prosthetic heart valve within the native heart valve with the actuator assembly and retracting the actuator assembly away from the radially expanded prosthetic heart valve.

In some examples, a method comprises advancing a distal end portion of a delivery apparatus toward a native heart valve, the delivery apparatus releasably coupled to a prosthetic heart valve by at least one actuator assembly, the prosthetic heart valve retained by the delivery apparatuses in a radially compressed state and comprising a frame comprising at least one actuation mechanism, the at least one actuation mechanism comprising a first frame member, a second frame member spaced axially apart from the first frame member, and an actuator extending through the first frame member and the second frame member, wherein the at least one actuator assembly comprises an outer sleeve having a tube portion abutting an outflow end portion of the second frame member and a first extension that extends distally from the tube portion and over the second frame member, a driver extending through the tube portion of the outer sleeve and engaging an end of the actuator, and a retaining element extending through aligned apertures in the outer sleeve and the second frame member such that the outer sleeve is retained against the outflow end portion of the second frame member. The method further comprises rotating the driver to cause corresponding rotation of the actuator to radially expand the prosthetic heart valve within the native heart valve while the outer sleeve is retained against the outflow end portion of the second frame member via the retaining element. The method further comprises releasing the retaining element from the second frame member and outer sleeve such that the outer sleeve and the second frame member are uncoupled from one another; and retracting the actuator assembly away from the prosthetic heart valve.

In some examples, a method comprises one or more of the features recited in Examples 50-60 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic device, according to an example.

FIG. 1B is a side elevation view of a frame of the prosthetic device of FIG. 1A.

FIG. 2 is a side elevation view of a delivery apparatus for a prosthetic device, according to an example.

FIG. 3 is a perspective view of a portion of an actuator of the prosthetic device of FIGS. 1A-1B and an actuator assembly of a delivery apparatus, according to an example.

FIG. 4 is a perspective view of the actuator and actuator assembly of FIG. 3 with the actuator assembly physically coupled to the actuator.

FIG. 5 is a perspective view of the actuator and actuator assembly of FIG. 3 with an outer sleeve of the actuator assembly positioned over an end portion of the actuator.

FIG. 6 is a perspective view of an actuator assembly of a delivery apparatus, the actuator assembly including an outer sleeve and a retaining element removably coupling the outer sleeve to a frame of a prosthetic device, according to an example.

FIG. 7 is a cross-sectional perspective view of the actuator assembly of FIG. 6 showing the retaining element coupled to the outer sleeve and a post of the frame of the prosthetic device.

FIG. 8 is a cross-sectional side view of the actuator assembly of FIG. 6.

FIG. 9 is another cross-sectional perspective view of the actuator assembly of FIG. 6.

FIG. 10 is a cross-sectional end view of an actuator assembly of a delivery apparatus, the actuator assembly including an outer sleeve and a retaining element removably coupling the outer sleeve to a frame of a prosthetic device, according to an example.

FIG. 11 is a cross-sectional side view of the actuator assembly of FIG. 10.

FIG. 12A is a perspective view of an actuator assembly of a delivery apparatus, the actuator assembly including an outer sleeve and a retaining element configured to removably couple the outer sleeve to a frame of a prosthetic device, according to an example.

FIG. 12B is a perspective view of the actuator assembly of FIG. 12A in a second configuration.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

Prosthetic devices disclosed herein can be advanced through a patient's vasculature on delivery apparatuses. The prosthetic devices can include one or more expansion and locking mechanisms that can be actuated using the delivery apparatuses to radially expand the prosthetic device and lock the prosthetic devices in one or more radially expanded states. As one example, the prosthetic devices can be crimped on or retained by the delivery apparatuses in a radially compressed state during delivery, and then radially expanded (and axially shortened) to a radially expanded state once the prosthetic devices reach the implantation site. It is understood that the prosthetic devices disclosed herein may be used with a variety of implant delivery apparatuses, and examples thereof will be discussed in more detail later.

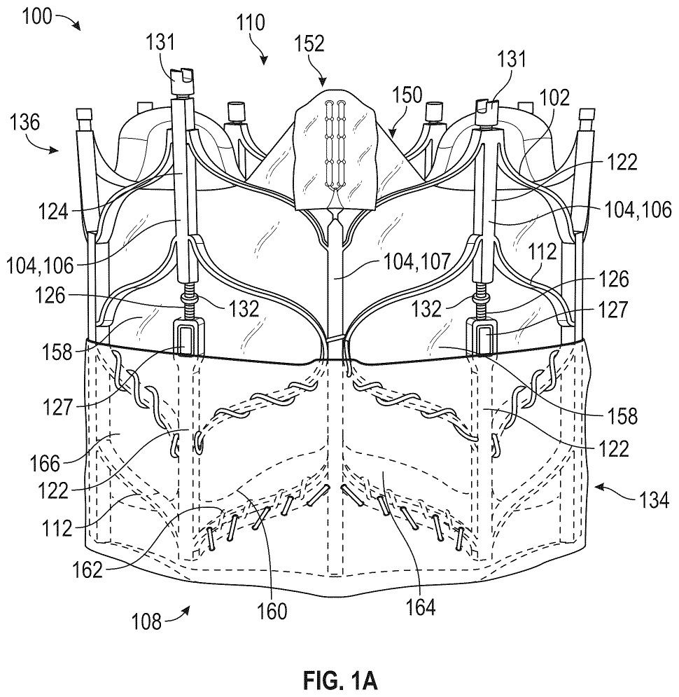

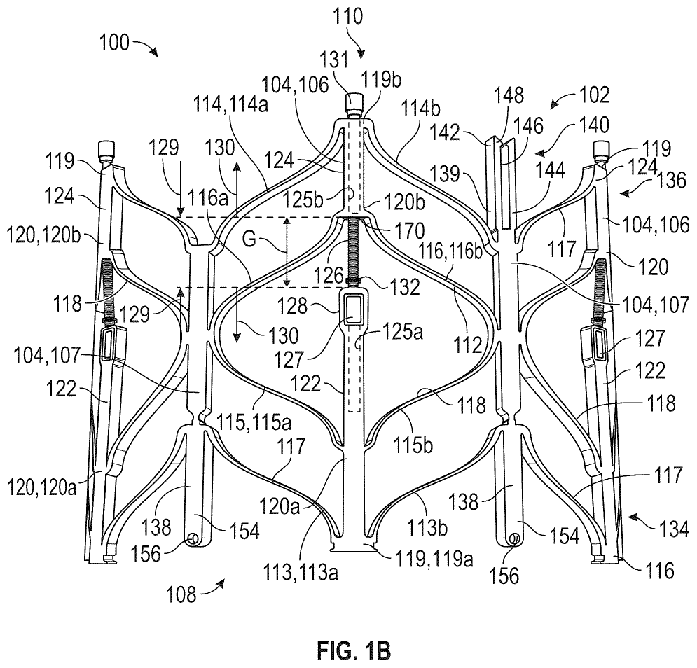

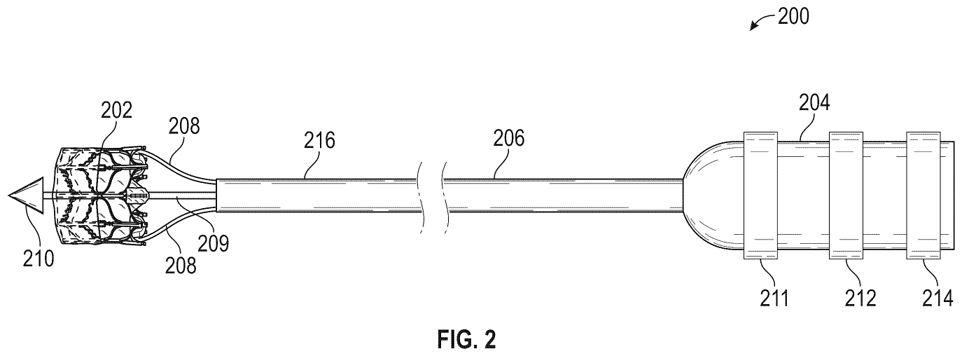

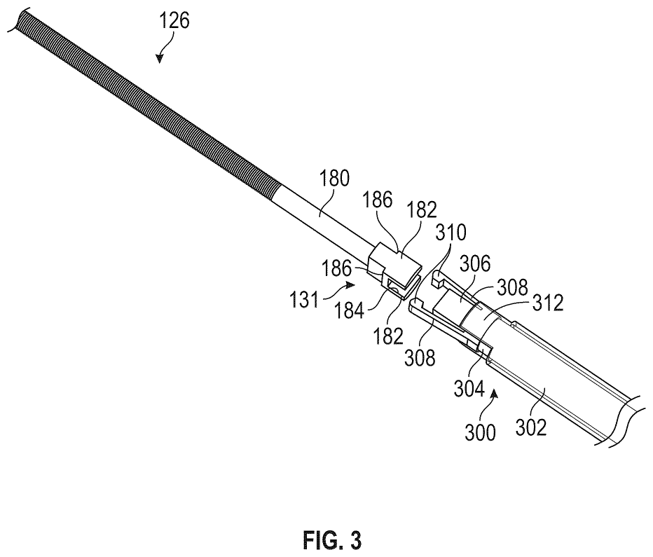

FIGS. 1A-1B illustrate an exemplary prosthetic device (e.g., prosthetic heart valve) that can be advanced through a patient's vasculature, such as to a native heart valve, by a delivery apparatus, such as the exemplary delivery apparatus shown in FIG. 2. The frame of the prosthetic device can include mechanical expansion and locking mechanisms that can be integrated into the frame—specifically, into axially extending posts of the frame. The mechanical expansion and/or locking mechanisms can be removably coupled to, and/or actuated by, the delivery apparatus. Specifically, the mechanical expansion and/or locking mechanisms of the frame can be removably coupled to an actuator assembly (FIGS. 3-5) of the delivery apparatus, which in turn can be actuated by a physician by adjusting and/or manipulating one or more input devices (e.g., one or more knobs, buttons, drawstrings, etc.) that can be included on a handle of the delivery apparatus.

For example, the actuator assembly of the delivery apparatus can include a driver covered by an outer sleeve. The driver is configured to interface with and rotate a portion of a frame of the prosthetic device while the outer sleeve is pressed against another portion of the frame to provide a counter-force during rotation of the driver, thereby causing radial expansion of the frame of the prosthetic heart valve. If the sleeve is not immovably secured against the frame during actuation (e.g., rotation of the driver), the sleeve can become uncoupled from the frame and the prosthetic heart valve can rotate or move during expansion. Accordingly, a need exists for improved actuation assemblies and interfaces between a delivery apparatus and a mechanically expandable prosthetic heart valve that allow for more efficient expansion of the prosthetic heart valve.

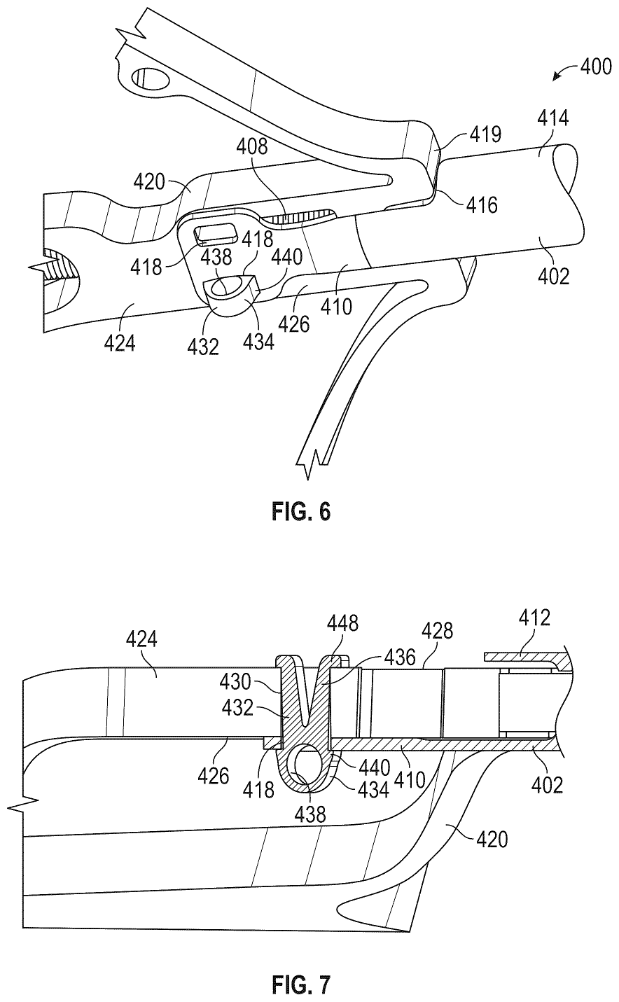

As illustrated in FIGS. 6-12B, various release mechanisms can be used to removably couple a sleeve of the actuator assembly of the delivery apparatus to a portion of the frame of the prosthetic device. As a result, during radial expansion of the frame of the prosthetic device, the actuator assembly can be rigidly held against the frame of the prosthetic device, thereby preventing rotation of the prosthetic device during expansion. Following expansion of the frame of the prosthetic device, the release mechanisms can be easily removed, thereby uncoupling the actuator sleeve(s) from the frame and enabling removal of the delivery apparatus from an implantation site.

### Examples of the Disclosed Technology

FIGS. 1A-1B illustrate an exemplary example of a prosthetic device 100 (which also may be referred to herein as “prosthetic heart valve 100” and/or “prosthetic valve 100”) having a frame 102. FIG. 1B shows the frame 102 by itself, while FIG. 1A shows the frame 102 with an optional valvular structure 150 (which can comprise leaflets 158, described further below) and an optional skirt assembly. While only one side of the frame 102 is depicted in FIG. 1B, it should be appreciated that the frame 102 forms an annular structure having an opposite side that is substantially identical to the portion shown in FIG. 1B.

The frame 102 comprises an inflow end 108, an outflow end 110, and a plurality of axially extending posts 104 extending therebetween. Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame, as further described below. One or more of posts 104 can be configured as support posts 107. The actuator mechanisms 106 (which can be used to radially expand and/or radially compress the prosthetic device 100) can be integrated into the frame 102 of the prosthetic device 100, thereby reducing the crimp profile and/or bulk of the prosthetic device 100. Integrating the actuator mechanisms 106 (which can also be referred to herein as “expansion and locking mechanisms”) into the frame 102 can also simplify the design of the prosthetic device 100, making the prosthetic device 100 cheaper and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic device 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (i.e., each strut 112 can be coupled on one end to one of the posts 122, 124 and can be coupled on the other end to one of the support posts 107). However, the prosthetic device 100 can include different numbers of support posts 107 and pairs of posts 122, 124 and/or the pairs of posts 122, 124 and the support posts 107 can be arranged in a non-alternating order, in some examples.

As illustrated in FIG. 1B, the struts 112 can include a first row of struts 113 at or near the inflow end 108 of the prosthetic device 100, a second row of struts 114 at or near the outflow end 110 of the prosthetic device 100, and third and fourth rows of struts 115, 116, respectively, positioned axially between the first and second rows of struts 113, 114. The struts 112 can form and/or define a plurality of cells (i.e., openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIG. 1B, each second cell 118 can be disposed within one of the first cells 117 (i.e., the struts 115a-116b forming the second cells 118 are disposed between the struts forming the first cells 117 (i.e., the struts 113a, 113b and the struts 114a, 114b), closer to an axial midline of the frame 102 than the struts 113a-114b).

As illustrated in FIG. 1B, the struts 112 of frame 102 can comprise a curved shape. Each first cell 117 can have an axially-extending hexagonal shape including first and second apices 119 (e.g., an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (e.g., distal apex 120a and proximal apex 120b). In some examples, the frame 102 comprises six first cells 117 extending circumferentially in a row, six second cells 118 extending circumferentially in a row within the six first cells 117, and twelve posts 104.

However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame and are axially separated from one another by a gap G (FIG. 1B) (those with actuators 126 can be referred to as actuator posts or actuator struts). Each first post 122 (i.e., the lower post shown in FIG. 1B) can extend axially from the inflow end 108 of the prosthetic device 100 toward the second post 124, and the second post 124 (i.e., the upper post shown in FIG. 1B) can extend axially from the outflow end 110 of the prosthetic device 100 toward the first post 122. For example, each first post 122 can be connected to and extend from an inflow apex 119a and each second post 124 can be connected to and extend from an outflow apex 119b. Each first post 122 can include an inner bore 125a and each second post 124 can include an inner bore 125b (FIG. 1B). The bores 125a, 125b of each pair of actuator posts 122, 124 can receive an actuator member, such as in the form of a substantially straight threaded rod or bolt 126 as shown in the illustrated example. The threaded rod 126 also may be referred to herein as actuator 126, actuator member 126, and/or screw actuator 126. In examples where the delivery apparatus can be releasably connected to the outflow end of the frame, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore 125b of the second post 124 (through which a rod 126 extends) can have a smooth and/or non-threaded inner surface to allow the rod 126 to slide freely within the inner bore 125b. Rotation of the threaded rod 126 relative to the nut 127 produces radial expansion and compression of the frame 102, as further described below.

In some examples, the threaded rod 126 can extend past the nut 127 toward the inflow end of the frame into the inner bore 125a of the first post 122. The nut 127 can be held in a fixed position relative to the first post 122 such that the nut 127 does not rotate relative to the first post 122. In this way, whenever the threaded rod 126 is rotated (e.g., by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the post 122 unless the threaded rod 126 is rotated relative to the nut 127. Thus, the threaded rod 126 can be retained or held by the nut 127 and can only be moved relative to the nut 127 and/or the post 122 by rotating the threaded rod 126 relative to the nut 127 and/or the post 122.

In some examples, in lieu of using the nut 127, at least a portion of the inner bore 125a of the first post 122 can be threaded. For example, the bore 125a along the end portion 128 of the first post 122 can comprise inner threads that engage the external threads of the threaded rod 126 such that rotation of the threaded rod causes the rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic device 100 can include six pairs of posts 122, 124, and each of the six pairs of posts 122, 124 with their corresponding rods 126 can be configured as one of the expansion and locking mechanisms 106 for a total of six expansion and locking mechanisms 106.

In some examples, not all pairs of posts 122, 124 need be expansion and locking mechanisms (i.e., actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic device 100). Specifically, when the threaded rod 126 is rotated relative to the nut 127, the first post 122, and the second post 124, the first and second posts 122, 124 can move axially relative to one another, thereby widening or narrowing the gap G (FIG. 1B) separating the posts 122, 124, and thereby radially compressing or radially expanding the prosthetic device 100, respectively. Thus, the gap G (FIG. 1B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, as will be described in greater detail below with reference to FIGS. 3-5, the head portion 131 can removably or releasably couple the threaded rod 126 to a respective actuator assembly of a delivery apparatus that can be used to radially expand and/or radially compress the prosthetic device 100. Second, the head portion 131 can prevent the second post 124 from moving proximally relative to the threaded rod 126 and can apply a distally directed force to the second post 124, such as when radially expanding the prosthetic device 100. Specifically, the head portion 131 can have a width greater than a diameter of the inner bore 125b of the second post 124 such that the head portion 131 is prevented from moving into the inner bore 125b of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic device 100.

The threaded rod 126 also can include a stopper 132 (e.g., in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (e.g., clockwise) can cause corresponding axial movement of the first and second posts 122, 124 toward one another (as shown by arrows 129 in FIG. 1B), thereby radially expanding the frame 102, while rotation of the threaded rod 126 in an opposite second direction (e.g., counterclockwise) causes corresponding axial movement of the first and second posts 122, 124 away from one another (as shown by arrows 130 in FIG. 1B), thereby radially compressing the frame. When the threaded rod 126 is rotated in the first direction, the head portion 131 of the rod 126 bears against an adjacent surface of the frame (e.g., an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 110 of the frame until the stopper 132 abuts the inflow end 170 of the second post 124. Upon further rotation of the rod 126 in the second direction, the stopper 132 can apply a proximally directed force to the second post 124 to radially compress the frame 102. Specifically, during crimping/radial compression of the prosthetic device 100, the threaded rod 126 can be rotated in the second direction (e.g., counterclockwise) causing the stopper 132 to push against (i.e., provide a proximally directed force to) the inflow end 170 of the second post 124, thereby causing the second post 124 to move away from the first post 122, and thereby axially elongating and radially compressing the prosthetic device 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic device 100 while the stopper 132 can apply a proximally directed force to the second post 124 to radially compress the prosthetic device 100. As explained above, radially expanding the prosthetic device 100 axially foreshortens the prosthetic device 100, causing an inflow end portion 134 and outflow end portion 136 of the prosthetic device 100 to move towards one another axially, while radially compressing the prosthetic device 100 axially elongates the prosthetic device 100, causing the inflow and outflow end portions 134, 136 to move away from one another axially.

In some examples, the rod 126 can be fixed against axial movement relative to the second post 124 (and the stopper 132 can be omitted) such that rotation of the rod 126 in the first direction produces proximal movement of the nut 127 and radial expansion of the frame and rotation of the rod 126 in the second direction produces distal movement of the nut 127 and radial compression of the frame 102.

As also introduced above, some of the posts 104 can be configured as support posts 107. As shown in FIG. 1B, the support posts 107 can extend axially between the inflow and outflow ends 108, 110 of the frame 102 and each can have an inflow end portion 138 and an outflow end portion 139. The outflow end portion 139 of one or more support posts 107 can include a commissure support structure or commissure support member 140. The commissure support member 140 can comprise first and second commissure arms 142, 144 defining a commissure opening 146 between them. The outflow end of each commissure arm 142, 144 can include a tooth 148 extending into the commissure opening 146.

The commissure opening 146 can extend radially through a thickness of the support post 107 and can be configured to accept a portion of a valvular structure 150 (e.g., a commissure 152) to couple the valvular structure 150 to the frame 102. For example, each commissure 152 can be mounted to a respective commissure support member 140, such as by inserting a pair of commissure tabs of adjacent leaflets through the opening 146 and suturing the commissure tabs to each other and/or the arms 142, 144. In some examples, the opening 146 can be fully enclosed by the support post 107 (e.g., not extending to the outflow edge) such that a portion of the valvular structure 150 can be slid radially (rather than axially) into the commissure opening 146 during assembly. The teeth 148 can help retain the commissure 152 within the commissure opening 146. In the illustrated example, the commissure opening 146 has a substantially rectangular shape and extends to the distal end of the post 104.

However, in some examples, the commissure opening can have any of various shapes (e.g., square, oval, square-oval, triangular, L-shaped, T-shaped, C-shaped, etc.).

Though only one support post 107 comprising a commissure support member 140 is shown in FIG. 1B, it should be noted that the frame 102 can comprise any number of support posts 107, any number of which can be configured as commissure support members 140. For example, the frame 102 can comprise six support posts 107, three of which are configured as commissure support members 140.

However, in some examples, the frame 102 can comprise more or less than six support posts 107 and/or more or less than three commissure support members 140.

The inflow end portion 138 of each support post 107 can comprise an extension 154 that extends toward the inflow end 108 of the frame 102. Each extension 154 can comprise an aperture 156 extending radially through a thickness of the extension 154. In some examples, the extension 154 can extend such that an inflow edge of the extension 154 aligns with or substantially aligns with the inflow end of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts can be coupled. For example, sutures used to connect the inner and/or outer skirts can be wrapped around the extensions 154 and/or can extend through apertures 156.

The frame 102 can be a unitary and/or fastener-free frame that can be constructed from a single piece of material (e.g., Nitinol, stainless steel, or a cobalt-chromium alloy), such as in the form of a tube. The plurality of cells can be formed by removing portions (e.g., via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic device 100 can be placed in a radially compressed state along the distal end portion of a delivery apparatus for insertion into a patient's body. When at the desired implantation site, the frame 102 (and therefore the prosthetic device 100) can be radially expanded from the radially compressed state to a radially expanded state via actuation of actuation assemblies of the delivery apparatus (as further described below), which rotate the rods 126 to produce expansion of the frame. During delivery to the implantation site, the prosthetic device can be placed inside of a delivery capsule (sheath) to protect against the prosthetic device contacting the patient's vasculature, such as when the prosthetic device is advanced through a femoral artery. The capsule can also retain the prosthetic device in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (e.g., Nitinol). When the frame 102 is formed from a self-expandable material, the prosthetic device can be radially compressed and placed inside the capsule of the delivery apparatus to maintain the prosthetic device in the radially compressed state while it is being delivered to the implantation site. When at the desired implantation site, the prosthetic device is deployed or released from the capsule. In some examples, the frame (and therefore the prosthetic device) can partially self-expand from the radially compressed state to a partially radially expanded state. The frame 102 (and therefore the prosthetic device 100) can be further radially expanded from the partially expanded state to a further radially expanded state via actuation of actuation assemblies of the delivery apparatus (as further described below), which rotate the rods 126 to produce expansion of the frame.

As illustrated in FIG. 1A, the prosthetic device 100 can further include the valvular structure 150, which is coupled to and supported inside the frame 102. The valvular structure 150 is configured to regulate the flow of blood through the prosthetic device 100, from the inflow end to the outflow end. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form the commissures 152, each of which can be secured to a respective commissure support member 140 and/or to other portions of the frame 102.

In the example depicted in FIG. 1A, the valvular structure 150 includes three leaflets 158, which can be arranged to collapse in a tricuspid arrangement. Each leaflet 158 can have an inflow or cusp edge portion 160. As shown in FIG. 1A, the inflow edge portions 160 of the leaflets 158 can define an undulating, curved scallop edge that generally follows or tracks portions of the struts 112 of frame 102 in a circumferential direction when the frame 102 is in the radially expanded configuration. The inflow edge portions 160 of the leaflets can be referred to as a “scallop line.”

As shown in FIG. 1A, the inflow edge portions 160 of the leaflets 158 can be sutured to an inner skirt 164 generally along the scallop line. The inner skirt 164 can in turn be sutured, via one or more sutures 162, for example, to adjacent struts 112 of the frame 102.

In some examples, the leaflets 158 can be sutured directly to the frame 102 along the scallop line.

The prosthetic device 100 can further include one or more skirts or sealing members. For example, the prosthetic device 100 can include the inner skirt 164, mounted on the radially inner surface of the frame 102. The inner skirt 164 can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic device 100.

The prosthetic device 100 can further include an outer skirt 166 mounted on the outer surface of the frame 102. The outer skirt 166 can be secured to the frame, such as with sutures 168 extending through the skirt 166 and around selected struts 112 of the frame. As noted above, the inflow edge portion of the outer skirt 166 optionally can be secured to the extensions 154, such as with sutures that extend through the apertures 156 and the skirt 166. The outer skirt 166 can function as a sealing member for the prosthetic device 100 by sealing against the tissue of the native valve annulus and helping to reduce paravalvular leakage past the prosthetic device 100.

The inner and outer skirts 164, 166 can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Application Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021 and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

As introduced above, the threaded rods 126 can removably couple the prosthetic device 100 to actuator assemblies of a delivery apparatus. Referring to FIG. 2, it illustrates an exemplary delivery apparatus 200 for delivering a prosthetic device or valve 202 (e.g., prosthetic device 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, an elongated first shaft 206 (which comprises an outer shaft in the illustrated example) extending distally from the handle 204, at least one actuator assembly 208 extending distally through the first shaft 206, an elongated second shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (e.g., actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery.

The actuator assemblies 208 can be releasably coupled to the prosthetic valve 202. For example, in the illustrated example, each actuator assembly 208 can be coupled to a respective actuator (e.g., threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the first shaft 206 or through separate respective lumens formed in the first shaft 206.

The handle 204 of the delivery apparatus 200 can include one or more control mechanisms (e.g., knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (e.g., clockwise) can retract the sheath 216 proximally relative to the prosthetic valve 202 and rotation of the first knob 211 in a second direction (e.g., counter-clockwise) can advance the sheath 216 distally. In some examples, the first knob 211 can be actuated by sliding or moving the knob 211 axially, such as pulling and/or pushing the knob. In some examples, actuation of the first knob 211 (rotation or sliding movement of the knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208, as will be described in greater detail below with reference to FIGS. 3-5. Rotation of the second knob 212 in a first direction (e.g., clockwise) can radially expand the prosthetic valve 202 and rotation of the second knob 212 in a second direction (e.g., counter-clockwise) can radially collapse the prosthetic valve 202. In some examples, the second knob 212 can be actuated by sliding or moving the knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (e.g., threaded rod), as further described below. Once the actuator assemblies 208 are uncoupled from the prosthetic valve 202, the delivery apparatus 200 can be removed from the patient, leaving just the prosthetic valve 202 in the patient.

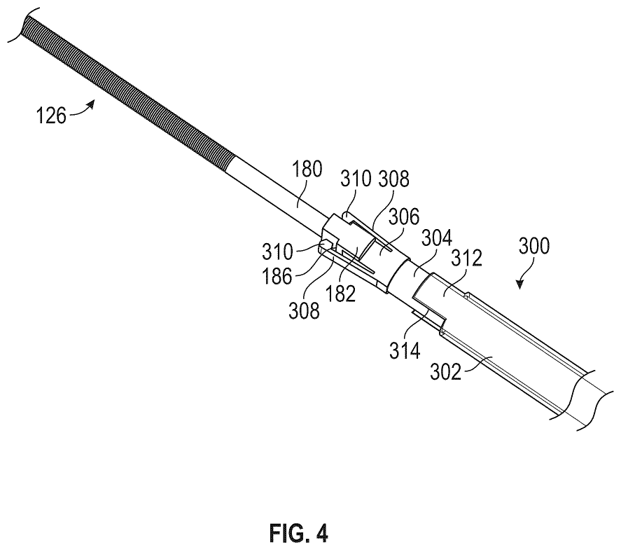

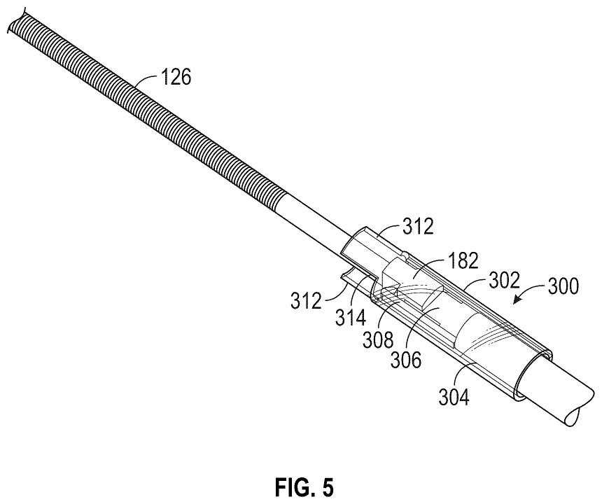

Referring to FIGS. 3-5, an exemplary actuator assembly 300 (e.g., actuator assemblies 208) of a delivery apparatus (e.g., delivery apparatus 200) configured to be removably coupled to an actuator or threaded rod 126 of the prosthetic device 100 is shown. Specifically, FIGS. 4 and 5 illustrate how one of the threaded rods 126 of the prosthetic device 100 can be coupled to an actuator assembly 300, while FIG. 3 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 1B). The head portion 131 can comprise first and second protrusions 182 defining a channel or slot 184 between them, and one or more shoulders 186. As discussed above, the head portion 131 can have a width greater than a diameter of the inner bore 125b (FIG. 1B) of the second post 124 (FIG. 1B) such that the head portion 131 is prevented from moving into the inner bore 125b of the second post 124 and such that the head portion 131 abuts the outflow end 110 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 3-5 for purposes of illustration. The distal end portions of the outer sleeve 302 and driver 304 can be configured to engage or abut the proximal end of the threaded rod 126 (e.g., the head portion 131) and/or the frame 102 (e.g., the apex 119b). The proximal portions of the outer sleeve 302 and driver 304 can be operatively coupled to the handle of a delivery apparatus (e.g., handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304.

In some examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the driver 304. As shown in FIGS. 3-4, the elongated elements 308 can be configured to be biased radially outward to an expanded state, for example, by shape setting the elements 308.

As shown in FIG. 4, to couple the actuator assembly 300 to the threaded rod 126, the driver 304 can be positioned such that the central protrusion 306 is disposed within the slot 184 (FIG. 3) and such that the protrusions 310 of the elongated elements 308 are positioned distally to the shoulders 186. As the outer sleeve 302 is advanced (e.g., distally) over the driver 304, the sleeve 302 compresses the elongated elements 308 they abut and/or snap over the shoulders 186, thereby coupling the actuator assembly 300 to the threaded rod 126 (FIG. 5). Thus, the outer sleeve 302 effectively squeezes and locks the elongated elements 308 and the protrusions 310 of the driver 304 into engagement with (i.e., over) the shoulders 186 of the threaded rod 126, thereby coupling the driver 304 to the threaded rod 126 (FIG. 5).

Because the central protrusion 306 of the driver 304 extends into the slot 184 of the threaded rod 126 when the driver 304 and the threaded rod 126 are coupled, the driver 304 and the threaded rod 126 can be rotationally locked such that they co-rotate. So coupled, the driver 304 can be rotated (e.g., using knob 212 the handle of the delivery apparatus 200) to cause corresponding rotation of the threaded rod 126 to radially expand or radially compress the prosthetic device. The central protrusion 306 can be configured (e.g., sized and shaped) such that it is advantageously spaced apart from the inner walls of the outer sleeve 302 (FIG. 5), such that the central protrusion 306 does not frictionally contact the outer sleeve 302 during rotation.

Though in the illustrated example the central protrusion 306 has a substantially rectangular shape in cross-section, in some examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308 (as shown in FIG. 5), until the outer sleeve 302 engages the frame 102 (e.g., a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (e.g., the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the second posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304 (as shown in FIG. 4). As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (e.g., knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

If the sleeve 302 of each actuator assembly 300 is not immovably secured against the frame (e.g., against the second post 124) during actuation (e.g., rotation of the driver 304), the sleeve 302 can become uncoupled from the frame. As a result, the prosthetic device can rotate or move during expansion.

Thus, a need exists for a mechanism that securely holds a sleeve of an actuator assembly (e.g., the sleeve 302) in engagement with a frame of a prosthetic device during radial expansion of the frame and also provides a releasable connection such that the sleeve can be easily disengaged from the frame after radial expansion is complete and the delivery apparatus is to be retracted from the implantation site.

FIGS. 6-9 show an example of a releasable engagement or retaining mechanism between a sleeve 402 (or actuation member) of an actuator assembly 400 and a frame 420 of a prosthetic device. In some examples, the frame 420 can be the same or similar to the frame 102 of the prosthetic device 100 of FIGS. 1A and 1B. Further, in some examples, the actuator assembly 400 can be similar to the actuator assembly 300 of FIGS. 3-5 and be part of a delivery apparatus, such as delivery apparatus 200 of FIG. 2.

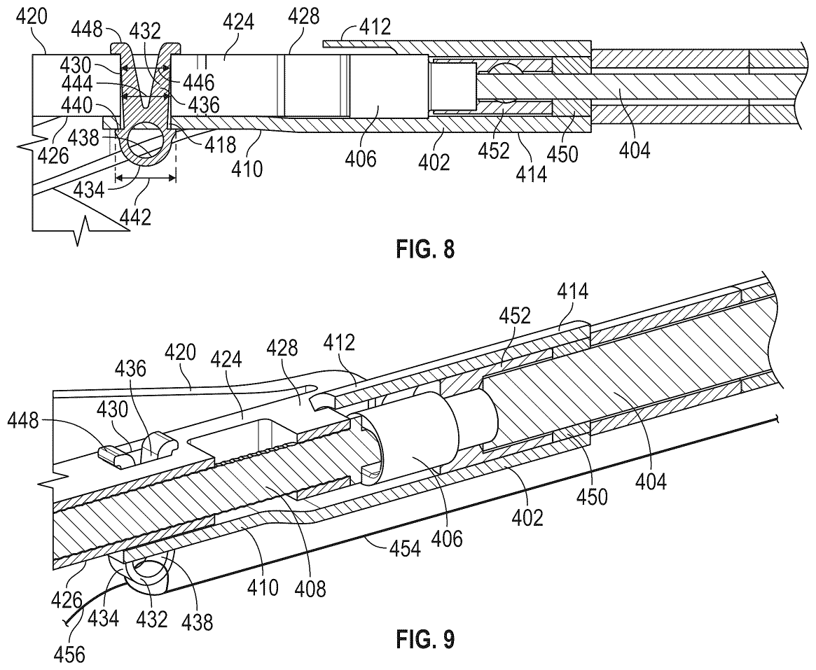

The actuator assembly 400 comprises a driver 404 (FIGS. 8 and 9) extending through and covered by the sleeve 402 (which can also be referred to as “outer sleeve 402”). The driver 404 comprises a driver head 406 at its distal end (FIGS. 8 and 9) that is configured to engage an end of a threaded rod 408 (or alternate actuator) of the frame 420 (FIGS. 6 and 9). Though the driver head 406 is depicted as a cup-shaped driver head 406 in FIGS. 8 and 9 and receives the end of the threaded rod 408 therein, in some examples, the driver head 406 can have a different configuration, such as a protrusion configured to engage the end of the threaded rod (e.g., the protrusion 306 of the driver 304 which engages the head portion 131 of the threaded rod 126 of the frame 102 shown in FIGS. 3-5).

A distal end portion of the sleeve 402 can comprise a first extension 410 and a second extension 412 which extend distally from a cylindrical or tube portion 414 of the sleeve 402 (similar to the extensions 312 of the outer sleeve 302 of FIGS. 3-5). However, the first extension 410 can be longer than and extend distal to the second extension 412 (FIGS. 7-9). When the actuator assembly 400 is coupled to the threaded rod 408, the first extension 410 can extend over a first side 426 of an end portion of a post 424 (which can also be referred to as a frame member) of the frame 420 (FIGS. 6-9) and the second extension 412 can extend over an opposite, second side 428 of the end portion of the post 424 (FIGS. 7-9). In some examples, the first side 426 can be an inner side (facing a central longitudinal axis of the frame 420), and thus, the first extension 410 can be referred to as an inner extension. In some examples, first side 426 can be an outer side (on an exterior of the frame relative to the central longitudinal axis of the frame 420).

The first extension 410 and the second extension 412 are spaced apart from one another in a circumferential direction and a radial (or lateral) direction. Thus, shoulders 416 are formed in the sleeve 402 at an end of the tube portion 414, between the first extension 410 and the second extension (e.g., the shoulders can be formed at the end of notches formed between the first and second extensions) (FIG. 6). The shoulders 416 can be positioned against an apex 419 of the frame (at the end of the post 424) when the actuator assembly 400 is coupled to the threaded rod 408.

A distal end portion of the first extension 410 can include one or more apertures 418 extending through a thickness of the first extension 410. Though two apertures 418 are shown in the first extension 410 in FIG. 6, in some examples the first extension 410 can only include a single aperture 418. The one or more apertures 418 can align with one or more corresponding apertures 430 in the post 424 of the frame 420 (FIGS. 7-9) when the actuator assembly 400 is coupled to the threaded rod 408. For example, the post 424 can comprise at least one aperture 430 offset (in a circumferential or lateral direction) from where the threaded rod 408 extends through the post 424, on one side of the threaded rod 408. The aperture 430 in the post 424 can extend through an entire thickness of the post 424, as shown in FIGS. 7-9.

A retaining element 432 can be configured to extend through the aperture 418 in the first extension 410 and the corresponding (and aligned) aperture 430 in the post 424 and releasably couple the first extension 410 of the sleeve 402 to the frame 420. As used herein, two components or apertures being “aligned” in a certain direction can refer to a straight line in that direction extending through the two aligned components or apertures.

In some examples, as shown in FIGS. 6-9, the retaining element 432 can be configured as a spring retainer comprising a head portion 434 and two spring-biased legs 436 extending from the head portion 434. In some examples, at least a portion of the head portion 434, such as a base 440 from which the legs 436 extend, can have a diameter or width 442 that is larger than a diameter or width 444 of the aperture 418 and aperture 430 (FIG. 8). As such, while the legs 436 of the retaining element 432 can extend through the aperture 418 and aperture 430, the head portion 434 can remain on an outside (or exterior) of the first extension 410.

The legs 436 can be configured to flex inward, toward one another, from a first (resting) orientation or position to a second orientation or position under external pressure. When the external pressure is removed, the legs 436 are configured to return or snap back into the first orientation. In this way, the legs 436 can be referred to herein as being “biased” or “spring biased”.

In some examples, the legs 436 and/or an entirety of the retaining element 432 can comprise a flexible material, such as Nitinol or another flexible metal or polymeric material.

In some examples, a distance between outer surfaces of the legs 436 (at a distal end portion of the legs 436—as shown by distance 446 in FIG. 8) when in the first orientation is greater than the width 444 of the aperture 430. Thus, when the legs 436 are arranged within the aperture 430 (as shown in FIGS. 7 and 8), the legs 436 exert an outward pressure against a wall of the post 424 defining the aperture 430.

To couple the retaining element 432 to the sleeve 402 and frame 420, the legs 436 can be pressed or pinched slightly together and pushed through the aperture 418 and the aperture 430 until the base 440 is positioned against and/or adjacent the first extension 410 and ends 448 of the legs 436 extend out of the aperture 430. In this position, the legs 436 can press against an inner surface of the post 424 that defines the aperture 430, thereby holding the retaining element 432 within the aperture 418 and the aperture 430.

In some examples, the ends 448 of the legs 436 can be angled or bent relative to a remainder of the legs 436 such that the bent ends 448 prevent the retaining element 432 from being inadvertently pulled out of the aperture 430 and disengaged from the frame 420. As shown in FIGS. 7-9, the bent ends 448 of the legs 436 can extend in an axial direction across a portion of the second side 428 of the post 424 disposed adjacent to the aperture 430.

In this way, the retaining element 432 can engage the post 424 of the frame 420 and hold the sleeve 402 against and in engagement with the frame. Thus, while the driver 404 actuates rotation of the threaded rod 408 (e.g., to radially expand the frame 420, as described above with reference to FIGS. 1A-5), the sleeve 402 can be held firmly against the frame 420, thereby preventing the frame 420 from rotating or translating during the radial expansion of the frame 420.

Additionally, an inner step 450 of the sleeve 402 can prevent the driver 404 from sliding proximally (toward a handle of the delivery apparatus) while the driver head 406 is engaged with the threaded rod 408 and the threaded rod 408 is being rotated by the driver 404. For example, as shown in FIGS. 8 and 9, the sleeve 402 can comprise the inner step 450 that extends radially inward from an inner circumferential surface of the tube portion 414 of the sleeve 402. The driver 404 can include an axial extension 452 that extends proximally from the driver head 406 toward the inner step 450.

Thus, the inner step 450 can maintain the driver head 406 engaged with the threaded rod 408 and the retaining element 432 can maintain the sleeve 402 engaged with the frame 420 during actuation (rotation) of the threaded rod 408 via the driver 404, thereby providing efficient radial expansion of the frame 420.

The head portion 434 can include an eyelet or aperture 438 which can be configured to receive a flexible member 454, such as a suture, cable, wire, or the like, that extends to a handle of the delivery apparatus (FIG. 9). For example, the flexible member 454 can be coupled to a control mechanism of a handle of the delivery apparatus (e.g., handle 204 of the delivery apparatus 200 of FIG. 2) and extend from the handle to the retaining mechanism and then be secured to (e.g., looped or tied around) the aperture 438 of the retaining element 432 (FIG. 9).

Actuation of the corresponding control mechanism on the handle can cause pulling of the flexible member 454 in a proximal direction (toward the handle), thereby causing the retaining element 432 to be pulled out of the apertures 418 and 430 and disengage from the frame 420 and the sleeve 402, thereby allowing the actuator assembly 400 to be uncoupled from the frame 420 after radial expansion of the frame 420 is complete.

In some examples, the control mechanism coupled to the flexible member 454 can be an additional rotatable knob on the handle (similar to knobs 211, 212, and/or 214) and/or another button or level on the handle.

In some examples, the control mechanism coupled to the flexible member 454 can be the third knob 214 and actuation of the third knob 214 can first result in release of the retaining element(s) 432 and then retraction of the sleeve 402 away from the frame of the prosthetic device.

In this way, rotation of the corresponding knob or pushing the corresponding button on the handle can result in disengagement of the retaining element 432 from the sleeve 402 and the frame 420 and then the delivery apparatus can be removed from the implantation site.

In some examples, each actuator assembly 400 of the delivery apparatus (when the frame 420 includes more than one actuator or threaded rod 408) can comprise a flexible member 454 connected between the retaining element 432 and the control mechanism of the handle of the delivery apparatus.

In some examples, the delivery apparatus can include one flexible member 454 connected to all of the actuator assemblies 400 of the delivery apparatus. For example, the flexible member 454 shown in FIG. 9 can be connected to the retaining element 432 and include an extension portion 456 that connects to an adjacent retaining element 432 of an adjacent actuator assembly of the delivery apparatus. Then, when pulling the flexible member 454 proximally, each retaining element 432 can be pulled away from its corresponding sleeve 402 in a serial manner, one after another, until all retaining elements (one for each actuator assembly) are released.

In some examples, portions of the legs 436 extending from the bent ends 448 can be angled such that the legs 436 can easily slide through and away from the apertures 430 and 418 when the flexible member 454 pulls against the head portion 434 of the retaining element 432. In some examples, an angle between the bent ends 448 and the portions of the legs 436 disposed adjacent to (and extending from) the bent end 448 can be selected such that the bent ends 448 can more easily slide through the aperture 430. For example, in some examples this angle can be greater than 90 degrees such that surfaces of the bent ends 448 facing the second side 428 of the post 424 can be angled away from and not in complete contact with the second side 428.

In some examples, when the first extension 410 is arranged against the first side 426 of the post 424, which is an inner side (or surface) of the post 424, a pulling force applied to the head portion 434 via the flexible member 454 results in pulling the retaining element 432 in a radially inward direction (toward a central longitudinal axis of the frame 420). This radially inward pulling force can result in minimal to no impact on the frame 420.

In some examples, at least a portion of the retaining element 432 can be radiopaque and comprise a radiopaque material (such as tantalum, bismuth, gold, iodine, barium, and/or the like). As a result, the retaining elements 432 of the delivery apparatus can be visible under fluoroscopy, thereby allowing a user (e.g., a clinician) to track the release of the retaining elements 432 prior to retrieval of the delivery apparatus. Thus, the user can ensure all retaining elements 432 are released from the frame 420 prior to removing the delivery apparatus from the implantation site and the patient. In some examples, a radiopaque marker can be embedded within a portion of the retaining element 432, such as the head portion 434.

In this way, the retaining element 432 can provide a secure but removable connection between the sleeve 402 of the actuator assembly 400 and the frame 420.

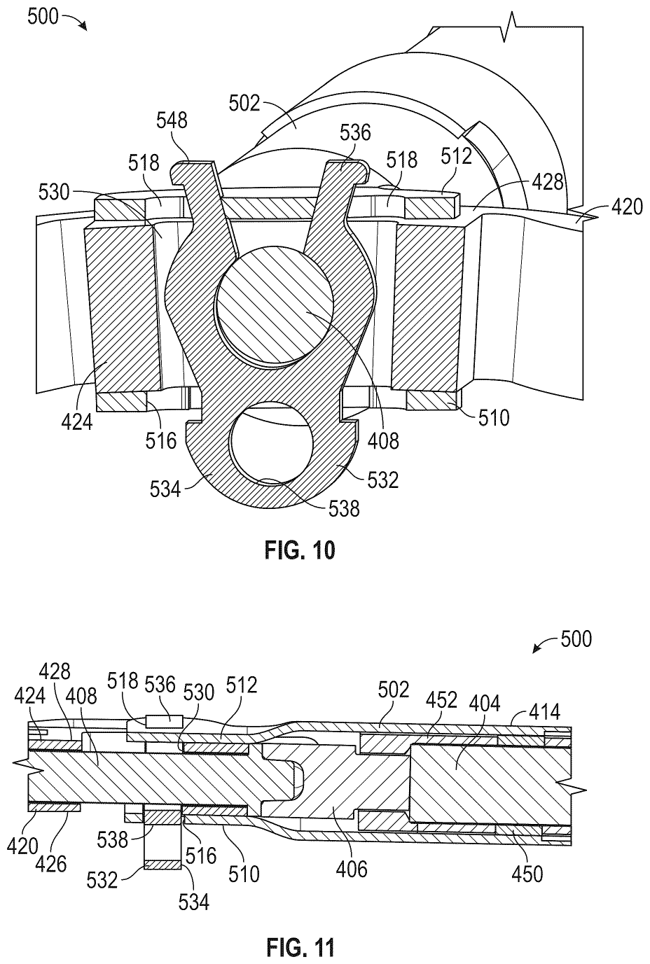

FIGS. 10 and 11 show an example of a releasable engagement or retaining mechanism between a sleeve 502 of an actuator assembly 500 and a frame 420 of a prosthetic device. The actuator assembly 500 can be similar to the actuator assembly 400 of FIGS. 6-9, except the sleeve 502 can comprise two extensions of a similar length that are both configured to engage a retaining element 532 (rather than just the first extension 410 of actuator assembly 400) in order to secure the sleeve 502 to the post 424 of the frame. The actuator assembly 500 can be part of a delivery apparatus, such as delivery apparatus 200 of FIG. 2.

Similar to the actuator assembly 400 (FIGS. 6-9), the actuator assembly 500 comprises a driver 404 extending through and covered by the sleeve 502 and configured engage an end of the threaded rod 408 (or alternate actuator) of the frame 420 (FIG. 11).

A distal end portion of the sleeve 502 can comprise a first extension 510 and a second extension 512 (FIG. 11) which extend distally from a cylindrical or tube portion 414 of the sleeve 502 (similar to the extensions 312 of the outer sleeve 302 of FIGS. 3-5). The first extension 510 and the second extension 512 can have an axial length such that when the actuator assembly 500 is coupled to the threaded rod 408, the first extension 510 and second extension 512 extend over the first side 426 and the second side 428 of the post 424, respectively, and over an aperture 530 in the post 424 that is configured to receive a retaining element 532 (FIG. 11), as described further below.

In some examples, the first side 426 can be an inner side (facing a central longitudinal axis of the frame 420) and the second side 428 can be an outer side (part of an exterior of the frame 420), and thus, the first extension 510 can be referred to as an inner extension and the second extension 512 can be referred to as an outer extension.

In some examples, first side 426 can be an outer side and the second side 428 can be an inner side of the frame 420.

The actuator assembly 500 includes a retaining element 532 configured to releasably couple the first extension 510 and the second extension 512 of the sleeve 502 to the post 424 of the frame 420. The first extension 510 incudes a first aperture 516 extending through a thickness of the first extension 510 and the second extension 512 includes two second apertures 518 spaced apart from one another and extending through a thickness of the second extension 512. When the actuator assembly 500 is engaged with the threaded rod 408, the first aperture 516 and the second apertures 518 align with the aperture 530 in the post 424. Said another way, the second apertures 518 can overlap with the aperture 530 and the aperture 516 in a radial direction.

The retaining element 532 can be configured as a spring retainer comprising a head portion 534 and two legs 536 extending from the head portion 534. The legs 536 can be flexible and comprise a flexible material, such as Nitinol or another flexible metal or polymeric material.

The first aperture 516 of the first extension 510 is sized to receive the retaining element 532 therethrough (from the legs 536 up to a base of the head portion 534) and the second apertures 518 of the second extension 512 are each sized to receive one of the legs 536 of the retaining element 532 therethrough. In some examples, as shown in FIG. 10, the legs 536 can have bent ends 548 that are angled or bent relative to a remainder of the legs 536. Thus, the second apertures 518 can be sized to receive the bent ends 548 therethrough. In some examples, the bent ends 548 can engage an outer surface of the second extension 512 and prevent the retaining element 532 from being inadvertently pulled out of engagement with the sleeve 502 and frame 420.

In some examples, the legs 536 may not include the bent ends 548.

In some examples, the bent ends 548 may not engage the outer surface of the second extension 512. Instead, the retaining element 532 can stay engaged with the post 424, in the radial direction, by being pressed around the threaded rod 408, as explained further below.

The aperture 530 in the post 424 is disposed around (circumferentially around) the threaded rod 408, as shown in FIG. 10. As such, the aperture 530 can be configured to receive the legs 536 of the retaining element 532 therethrough, with the legs 536 extending along both sides of the threaded rod 408 (e.g., one leg on each side of the threaded rod 408).

To couple the sleeve 502 to the post 424 of the frame using the retaining element 532, the legs 536 of the retaining element 532 can be inserted through the first aperture 516 in the first extension 510, through the aperture 530 in the post 424, and around the threaded rod 408. As the legs 536 engage the threaded rod 408 and are pushed around the threaded rod 408 (toward the second extension 512), the legs 536 are flexed outward (from their resting or first position or state), away from one another, and around the threaded rod 408. After passing over the threaded rod 408, the legs 536 spring back toward one another into or closer to their resting (unflexed) position. At the same time, the legs 536 can pass through respective second apertures 518 in the second extension 512. When the legs 536 spring back toward one another, they can press or squeeze against the threaded rod 408 and/or inner surfaces of the second apertures 518, thereby holding the first extension 510 and second extension 512 in engagement with the frame 420.

Thus, in contrast to the retaining element 432 of FIGS. 6-9, the retaining element 532 of FIGS. 10 and 11 comprises legs 536 that are configured to surround the threaded rod 408, with the threaded rod 408 disposed between the two legs 536 when the retaining element 532 is engaged with the sleeve 502 and the post 424, as described above. Further, in contrast to the retaining element 432 of FIGS. 6-9, the retaining element 532 of FIGS. 10 and 11 is configured to couple both sides of the sleeve 502 (the first extension 510 and the second extension 512) to the frame 420.

Additionally, the retaining element 532 can replace a stopper between the driver 404 and the threaded rod 408 (e.g., stopper 132 in FIGS. 1A and 1B) and prevent unintentional distal movement of the driver 404 during rotation. As discussed above, the inner step 450 of the sleeve 502 can also prevent unwanted proximal movement of the driver 404.

The head portion 534 of the retaining element 532 can also comprise an aperture 538. In some examples, the retaining element 532 can be connected to a handle of a delivery apparatus, in a same or similar manner as described above and shown in FIG. 9. In this way, the retaining element 532 can be configured to be removed, under a pulling force, from engagement with the sleeve 502 and frame 420, thereby allowing the actuator assembly 500 to be moved away from the frame 420 (and the delivery apparatus removed from the patient).

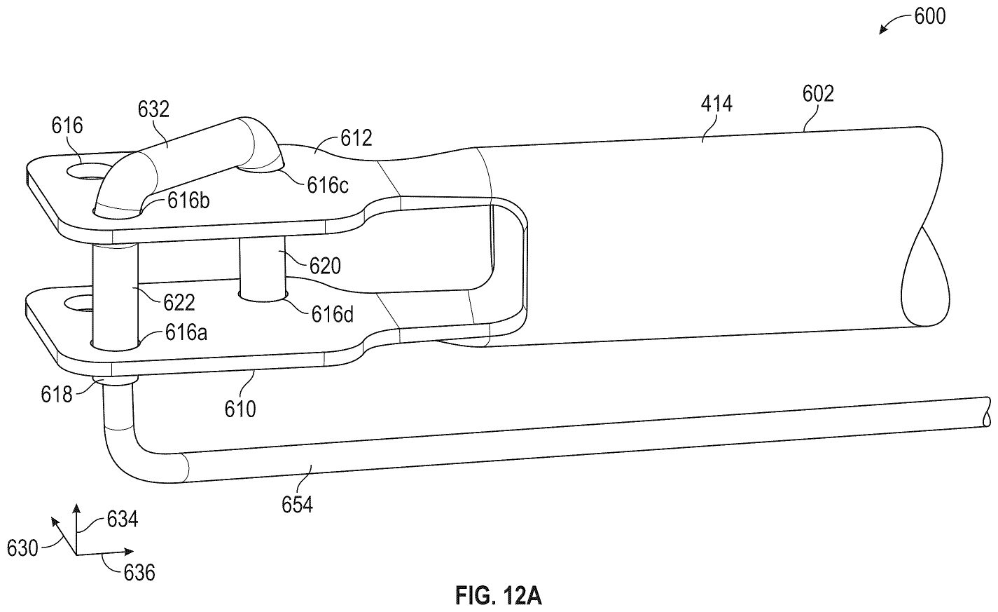

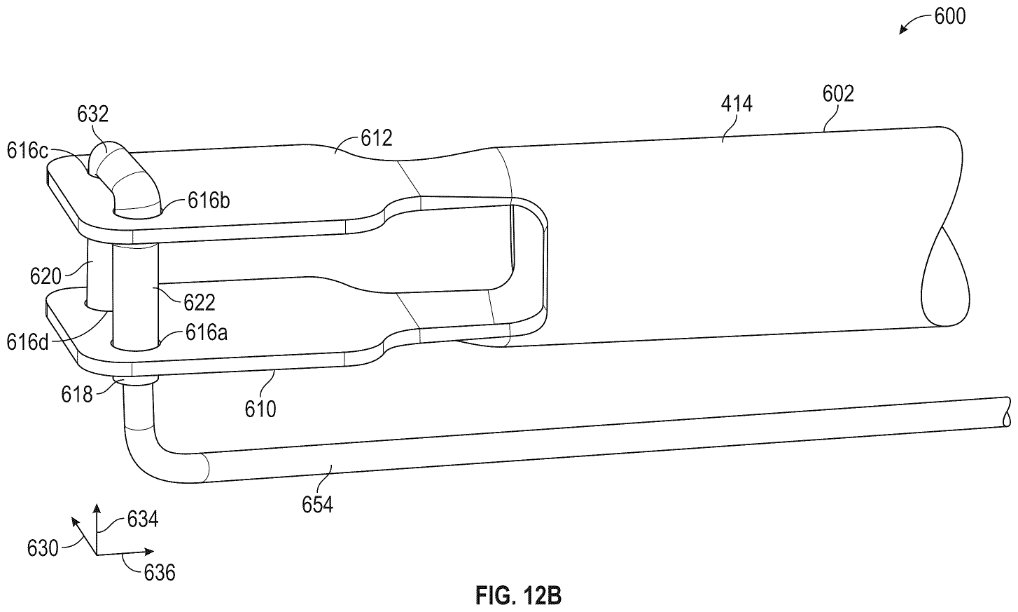

FIGS. 12A and 12B show an example of a releasable engagement or retaining mechanism between a sleeve 602 of an actuator assembly 600 and a frame of a prosthetic device. FIGS. 12A and 12B show a portion of the actuator assembly 600 including the sleeve 602 and a retaining element 632. However, the actuator assembly 600 can be similar to the actuator assembly 400 of FIGS. 6-9 and actuator assembly 500 of FIGS. 10 and 11 and include a driver (such as driver 404) configured to extend through the sleeve 602 and interface with a threaded rod of a prosthetic device, as described and shown herein. The sleeve 602 can be similar to the sleeve 502 of the actuator assembly 500 and include two extensions (first extension 610 and second extension 612) of a similar axial length that are both configured to engage the retaining element 632 in order to secure the sleeve 602 to a post of a frame of a prosthetic device (such as post 424 of frame 420 shown in FIGS. 6-11 or post 124 of frame 102 of FIGS. 1A and 1B). The actuator assembly 600 can be part of a delivery apparatus, such as delivery apparatus 200 of FIG. 2.

The first extension 610 and the second extension 612 can each include two or more apertures 616. FIG. 12A shows each of the first extension 610 and the second extension 612 having three apertures 616.

However, in some examples, the first extension 610 and the second extension 612 may only include two apertures 616 that are aligned in a circumferential direction 630 (as shown in FIG. 12B, a line extending in the circumferential direction 630 extends through both the apertures 616 in the same extension) or two apertures 616 that are offset both in the circumferential direction 630 and an axial direction 636 (e.g., the two apertures 616 that the retaining element 632 is passing through in FIG. 12A are spaced apart from one another both in the circumferential direction 630 and the axial direction 636). For reference, a coordinate axes showing the circumferential direction 630, axial direction 636, and radial (or lateral) direction 634 is depicted in FIGS. 12A and 12B.

The apertures 616 can be sized to receive the retaining element 632 therethrough, which can be configured as a flexible tubular element, as shown in FIGS. 12A and 12B.

In some examples, the retaining element 632 can be an elongate element having a different cross-sectional shape which corresponds to a shape of the apertures 616 (e.g., oblong, square, hexagonal, or the like).

The apertures 616 that are configured to receive the retaining element 632 can be offset from one another in the circumferential direction 630 such that when the actuator assembly 600 is coupled to a frame of the prosthetic device (e.g., driver 404 is engaged with the threaded rod 408, as shown in FIGS. 6-11) the opposing apertures 616 are aligned with corresponding apertures in the post of the frame of the prosthetic device that are disposed on opposite sides of the threaded rod 408. In this way, the post of the frame can also include an aperture disposed on both sides of the threaded rod that are configured to receive the retaining element 632 therethrough.

The retaining element 632 can comprise a formed but flexible material, such as wire or another flexible metal or polymeric material. As one example, the retaining element 632 can extend through a first aperture 616a in the first extension 610, extend through a second aperture 616b in the second extension 612 (the first aperture 616a and the second aperture 616b aligned in the radial direction 634), then be bent to extend across the second extension 612 to a third aperture 616c that is either unaligned in the circumferential direction 630 from the second aperture 616b (FIG. 12A) or aligned in the circumferential direction with the second aperture 616b (FIG. 12B). The retaining element 632 then bends again to extend through the third aperture 616c and across a distance separating the first extension 610 and the second extension 612 to and through a fourth aperture 616d in the first extension 610.

In this way, when the retaining element 632 is coupled with the sleeve 602 and a frame of a prosthetic heart valve (e.g., the second frame member or post 424, similar to as shown in FIGS. 6-11), the retaining element 632 can assume an approximately U-shaped configuration with a first leg 620 extending through opposing apertures 616c and 616d in the second extension 612 and the first extension 610, respectively, and corresponding apertures in the post of the frame of the prosthetic device (forming a first set of aligned apertures), and a second leg 622 extending through opposing apertures 616b and 616a in the second extension 612 and the first extension 610, respectively, and corresponding apertures in the post of the frame of the prosthetic device (forming a second set of aligned apertures). As a result, the retaining element 632 secures the sleeve 602 to the frame during radial expansion and/or compression of the frame of the prosthetic device.

Further, in this U-shaped configuration, the retaining element 632 has a free end extending through the fourth aperture 616d and an attached end 618 extending through the first aperture 616a and attached to a flexible element 654 that is connected to a handle of the delivery apparatus. Similar to as described above for flexible member 454 (FIG. 9), by pulling the flexible element 654, the retaining element 632 can be pulled through and away from the apertures 616d, 616c, 616b, and finally 616a. As a result, the sleeve 602 and the rest of the actuator assembly 600 can be uncoupled from the frame and removed from the implantation site.

The dimensions and material properties of the retaining element 632 can be selected such that the retaining element 632 retains the U-shaped configuration during radial expansion of the frame of the prosthetic device via the actuator assembly 600, thereby keeping the sleeve 602 attached to the frame, and also allows the retaining element 632 to straighten and be pulled through the apertures 616a-616d when pulled proximally by the flexible element 654 (e.g., due to actuation via a control mechanism at the handle of the delivery apparatus), thereby releasing the sleeve 602 from the frame.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for a radially expandable prosthetic device, comprising: an actuator assembly comprising: an outer sleeve comprising a tube portion and an extension that extends distally from the tube portion, the extension comprising a first aperture in its distal end; an actuation member extending through the tube portion of the outer sleeve; and a retaining element configured to extend through the first aperture in the extension of the outer sleeve and hold the outer sleeve against an end portion of a frame of a prosthetic device, wherein the retaining element is biased into a first position where it holds the outer sleeve in engagement with the frame, and is configured to flex in response to a pulling force into a second position that enables the retaining element to be released from the first aperture, thereby enabling disengagement of the outer sleeve from the end portion of the frame of the prosthetic device.

Example 2. The delivery apparatus of any example herein, particularly example 1, wherein the retaining element comprises a head portion including a second aperture and two legs spaced apart from one another and extending from the head portion, wherein the two legs are configured to flex between the first position and the second position.

Example 3. The delivery apparatus of any example herein, particularly example 2, wherein the extension of the outer sleeve is a first extension and wherein the outer sleeve comprises a second extension extending from an opposite side of the tube portion of the outer sleeve than the first extension, the first and second extensions spaced apart from one another.

Example 4. The delivery apparatus of any example herein, particularly example 3, wherein the first extension is longer than the second extension.

Example 5. The delivery apparatus of any example herein, particularly example 4, wherein in the first position the two legs are spaced further apart from one another than in the second position.

Example 6. The delivery apparatus of any example herein, particularly example 3, wherein the first extension and the second extension extend distally from the tube portion by a same amount, wherein the second extension comprises two second apertures that are spaced apart from one another, and wherein the first aperture of the first extension is configured to receive the two legs of the retaining element therethrough and each second aperture of the two second apertures of the second extension is configured to receive one of the two legs therethrough.

Example 7. The delivery apparatus of any example herein, particularly example 6, wherein in the second position the two legs are spaced further apart from one another than in the first position.

Example 8. The delivery apparatus of any example herein, particularly any one of examples 2-7, wherein each leg of the two legs comprises a bent end that is bent relative to a remainder of the leg, the bent end configured to further retain the retaining element within the first aperture and the outer sleeve in engagement with the frame.

Example 9. The delivery apparatus of any example herein, particularly any one of examples 2-8, wherein the head portion comprises a base from which the two legs extend, and wherein a width of the base is larger than a diameter of the first aperture such that the head portion remains exterior to the first aperture, on an outer surface of the extension.

Example 10. The delivery apparatus of any example herein, particularly example 1, wherein the extension of the outer sleeve is a first extension, wherein the outer sleeve comprises a second extension extending from an opposite side of the tube portion of the outer sleeve than the first extension, the first and second extensions spaced apart from one another, and wherein each of the first extension and the second extension include two apertures therein that are spaced apart from one another in a direction that is perpendicular to an axial direction of the actuator assembly.

Example 11. The delivery apparatus of any example herein, particularly example 10, the retaining element is a flexible tubular retaining element, and wherein in the first position the retaining element assumes a U-shaped configuration with a first leg of the retaining element extending through a first aperture of the two apertures of the first extension and a first aperture of the two apertures of the second extension, the first apertures of the first and second extensions opposing one another across a gap separating the first and second extensions, and a second leg extending through a second aperture of the two apertures in the first extension and a second apertures in the two apertures in the second extension, the second apertures opposing one another across the gap separating the first and second extensions.

Example 12. The delivery apparatus of any example herein, particularly example 11, wherein the retaining element comprises a free end at an end of the first leg and an attached end at an end of the second leg, the attached end coupled to a flexible element connected to a handle of the delivery apparatus, wherein the flexible element is configured to apply the pulling force to the attached end of the retaining element, via actuation from the handle, such that the retaining element flexes into the second position and is pulled out of the two apertures of each of the first extension and the second extension.

Example 13. The delivery apparatus of any example herein, particularly any one of examples 1-12, further comprising a handle, and wherein the actuator assembly extends distally from the handle.

Example 14. The delivery apparatus of any example herein, particularly example 13, further comprising a flexible element extending from the handle to an end portion of the retaining element and wherein the flexible element is configured to apply the pulling force to the end portion of the retaining element in response to actuation of a control mechanism of the handle.

Example 15. The delivery apparatus of any example herein, particularly any one of examples 1-14, wherein the outer sleeve further comprises an inner step extending radially inward from an inner circumferential surface of the tube portion, and wherein the actuation member comprises an end portion configured to engage an actuator of the prosthetic device and an axial extension extending proximally from the end portion of the actuation member toward the inner step.

Example 16. An assembly comprising: a prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another; and a threaded rod extending through the first and second inner bores; and a delivery apparatus comprising: a handle; at least one actuator assembly extending from the handle, the actuator assembly comprising: an outer sleeve having a tube portion abutting an outflow end portion of the second frame member and a first extension that extends distally from the tube portion and over the second frame member; a driver extending through the tube portion of the outer sleeve and engaging an end of the threaded rod, the driver configured to rotate the threaded rod to radially expand and/or compress the frame; and a retaining element extending through a first aperture in the first extension of the outer sleeve and a second aperture in the second frame member, the retaining element configured to hold the outer sleeve in engagement with the second frame member, and wherein the retaining element is configured to flex and release from the first and second apertures in response to a pulling force applied to an end portion of the retaining element.

Example 17. The assembly of any example herein, particularly example 16, wherein the retaining element comprises a head portion and two legs spaces apart from one another extending from the head portion, wherein the legs are configured to flex under external force toward or away from one another, and wherein the first aperture and the second aperture are shaped to receive the legs therethrough.

Example 18. The assembly of any example herein, particularly example 17, wherein the head portion comprises a third aperture, and wherein the third aperture is attached to a flexible element that extends to a control mechanism of the handle that is configured to pull the flexible element proximally toward the handle and remove the retaining element from the outer sleeve and the second frame member.

Example 19. The assembly of any example herein, particularly example 17 or example 18, wherein the first and second apertures are aligned with one another when the driver is coupled to the threaded rod, and wherein the second aperture extends through a thickness of the second frame member at a location offset from one side of the threaded rod.

Example 20. The assembly of any example herein, particularly example 19, wherein the legs are movable between a resting first position where the legs are pressed against the second aperture when the retaining element is coupled with the outer sleeve and the second frame member, and a flexed second position where the legs are pressed toward one another such that the legs can be released from the second aperture and the first aperture.

Example 21. The assembly of any example herein, particularly example 20, wherein end portions of the legs are bent relative to a remainder of the legs and extend out of the second aperture and across a portion of a surface of the second frame member when the retaining element is coupled with the outer sleeve and the second frame member and the legs are in the first position.

Example 22. The assembly of any example herein, particularly example 17 or example 18, wherein the first and second apertures are aligned with one another when the driver is coupled to the threaded rod, and wherein the second aperture extends through a thickness of the second frame member at a location of the second inner bore such that the threaded rod extends through the second aperture.

Example 23. The assembly of any example herein, particularly example 22, wherein the outer sleeve comprises a second extension that extends distally from the tube portion on an opposite side of the tube portion than the first extension, wherein the first and second extensions extend over opposite sides of the second frame member, and wherein the second extension comprises two fourth apertures spaced apart from one another and aligned with the first and second apertures, each fourth aperture of the two fourth apertures configured to receive one of the legs therethrough.

Example 24. The assembly of any example herein, particularly example 23, wherein when the retaining element extends through the first, second, and fourth apertures, the legs are disposed around the threaded rod, with one leg on each side of the threaded rod, and end portions of the legs extend through respective fourth apertures of the two fourth apertures and over an outer surface of the second extension.

Example 25. The assembly of any example herein, particularly example 24, wherein the end portions of the legs are bent relative to a remainder of the legs.

Example 26. The assembly of any example herein, particularly example 24 or example 25, wherein the legs are movable between a resting first position where the legs are pressed toward one another and against inner surfaces of the two fourth apertures and around the threaded rod and a flexed second position where the legs are pressed away from one another such that the legs can slide past the threaded rod and be released from the first, second and fourth apertures.

Example 27. The assembly of any example herein, particularly example 16, wherein the outer sleeve comprises a second extension extending from an opposite side of the tube portion of the outer sleeve than the first extension, the first and second extensions spaced apart from one another, wherein each of the first extension and the second extension include two apertures therein that are spaced apart from one another, and wherein the two apertures in the first extension are aligned with the two apertures in the second extension such that a first set of aligned apertures are formed in the first and second extensions and a second set of aligned apertures are formed in the first and second extensions.

Example 28. The assembly of any example herein, particularly example 27, wherein the retaining element is a flexible elongate retaining element that assumes a u-shape comprising two legs when the retaining element is coupled to the outer sleeve and the second frame member, wherein a first leg of the two legs extends through the first set of aligned apertures and a second leg of the two legs extends through the second set of aligned apertures.

Example 29. The assembly of any example herein, particularly example 28, wherein the retaining element comprises a free end extending through a first aperture of the two apertures in the first extension, and wherein the retaining element comprises an attached end disposed opposite the free end that extends through a second aperture of the two apertures in the first extension and is attached to a flexible element that extends to the handle.

Example 30. The assembly of any example herein, particularly any one of examples 16-29, wherein the handle comprises a first control mechanism configured to pull a flexible element extending from the handle and attached to the retaining element in a proximal direction, thereby applying the pulling force to the retaining element to remove the retaining element from the first and second apertures and disengage the outer sleeve and the second frame member.

Example 31. The assembly of any example herein, particularly example 30, wherein the handle comprises a second control mechanism configured to retract the outer sleeve away from the frame after removing the retaining element from the outer sleeve and the second frame member.

Example 32. An assembly comprising: a prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another; and a threaded rod extending through the first and second inner bores; and a delivery apparatus comprising: a handle; at least one actuator assembly extending from the handle, the actuator assembly comprising: an outer sleeve having a tube portion abutting an outflow end portion of the second frame member and a first extension that extends distally from the tube portion and over the second frame member, the first extension including a first aperture disposed in a distal end of the first extension; a driver extending through the tube portion of the outer sleeve and engaging an end of the threaded rod, the driver configured to rotate the threaded rod to radially expand the frame; and a retaining element releasably coupling the outer sleeve to the second frame member, the retaining element comprising a head portion and two legs extending distally from the head portion, wherein the two legs extend through the first aperture in the first extension and a second aperture in the second frame member and retain the outer sleeve in engagement with the second frame member.

Example 33. The assembly of any example herein, particularly example 32, wherein the first aperture of the first extension and the second aperture of the second frame member are aligned in a radial direction of the frame when the driver is engaged with the threaded rod and the outer sleeve abuts the outflow end portion of the second frame member.

Example 34. The assembly of any example herein, particularly example 32 or example 33, wherein the first extension extends over a radially inward facing surface of the second frame member, relative to a central longitudinal axis of the frame, and wherein the head portion of the retaining element is disposed on an outer surface of the first extension, exterior to the first aperture.

Example 35. The assembly of any example herein, particularly example 34, wherein the head portion comprises a base from which the two legs extend, and wherein a width of the base is larger than a diameter of the first aperture.

Example 36. The assembly of any example herein, particularly any one of examples 32-35, wherein the head portion comprises a third aperture and further comprising a flexible element extending from the handle and connected to the third aperture, wherein the flexible element is configured to exert a proximally directed force to the head portion via actuation of the handle such that the two legs of the retaining element are removed from the first and second apertures.

Example 37. The assembly of any example herein, particularly any one of examples 32-36, wherein the two legs are spaced apart from one another and configured to flex under a pulling force applied to the head portion from a first position where the retaining element couples the outer sleeve and the second frame member together and a second position where the two legs are released from the first aperture and the second aperture.

Example 38. The assembly of any example herein, particularly example 37, wherein the outer sleeve further comprises a second extension extending from an opposite side of the tube portion of the outer sleeve than the first extension, the first and second extensions spaced apart from one another in a radial direction relative to a central longitudinal axis of the frame.

Example 39. The assembly of any example herein, particularly example 38, wherein the first extension is longer than the second extension, and wherein the second aperture extends radially through the second frame member, offset to one side of the second inner bore through which the threaded rod extends.

Example 40. The assembly of any example herein, particularly example 38 or example 39, wherein the two legs are pressed against the second aperture in the first position, and wherein the two legs are flexed and disposed closer together in the second position than in the first position.

Example 41. The assembly of any example herein, particularly example 38, wherein the second extension comprises two fourth apertures that are spaced apart from one another and aligned with the first and second apertures when the outer sleeve abuts the outflow end portion of the second frame member, and wherein each of the fourth apertures receives one of the two legs of the retaining element therethrough when in the first position.

Example 42. The assembly of any example herein, particularly example 41, wherein in the two legs are pressed against inner surfaces of the fourth apertures and toward one another in the first position, and wherein the two legs are flexed away from one another in the second position.

Example 43. The assembly of any example herein, particularly any one of examples 32-42, wherein each leg of the two legs of the retaining element comprises a bent end that is angled relative to a remainder of the leg and configured to retain the two legs within the second aperture.

Example 44. The assembly of any example herein, particularly any one of examples 32-43, wherein the handle comprises a first control mechanism configured to pull a flexible element extending from the handle and attached to the retaining element in a proximal direction, thereby applying a pulling force to the retaining element to remove the retaining element from the first and second apertures and disengage the outer sleeve and the second frame member.

Example 45. The assembly of any example herein, particularly example 44, wherein the handle comprises a second control mechanism configured to retract the outer sleeve away from the frame after removing the retaining element from the outer sleeve and the second frame member.

Example 46. An assembly comprising: a prosthetic heart valve comprising: a radially expandable and compressible frame, the frame comprising at least one actuation mechanism comprising: a first frame member having a first inner bore and a second frame member having a second inner bore, the first and second frame members being spaced apart axially from one another; and a threaded rod extending through the first and second inner bores; and a delivery apparatus comprising: a handle; at least one actuator assembly extending from the handle, the actuator assembly comprising: an outer sleeve having a tube portion abutting an outflow end portion of the second frame member, first and second extensions that each extend distally from the tube portion and over the second frame member, the first and second extensions including two sets of aligned apertures; a driver extending through the tube portion of the outer sleeve and engaging an end of the threaded rod, the driver configured to rotate the threaded rod to radially expand the frame; and a flexible elongate retaining element releasably coupling the outer sleeve to the second frame member, the retaining element assuming a u-shape comprising two legs when the retaining element is coupled to the outer sleeve and the second frame member, wherein a first leg of the two legs extends through a first set of aligned apertures of the two sets of aligned apertures and a second leg of the two legs extends through a second set of aligned apertures of the two sets of aligned apertures such that the outer sleeve is retained in engagement with the second frame member.

Example 47. The assembly of any example herein, particularly example 46, wherein the two sets of aligned apertures are spaced apart from one another in a circumferential direction, and wherein the first and second extensions are spaced apart from one another in a radial direction.

Example 48. The assembly of any example herein, particularly example 46 or example 47, wherein the retaining element comprises a free end extending through a first aperture of the first set of aligned apertures that is disposed in the first extension, and wherein the retaining element comprises an attached end disposed opposite the free end that extends through a second aperture of the second set of aligned apertures that is disposed in the first extension and is attached to a flexible element that extends to the handle.

Example 49. The assembly of any example herein, particularly example 48, wherein the first extension extends over a radially inward facing surface of the second frame member, relative to a central longitudinal axis of the frame of the prosthetic heart valve.

Example 50. A method comprising: advancing a distal end portion of a delivery apparatus toward a native heart valve, the delivery apparatus releasably coupled to a prosthetic heart valve by at least one actuator assembly, the prosthetic heart valve retained by the delivery apparatuses in a radially compressed state and comprising a frame comprising at least one actuation mechanism, the at least one actuation mechanism comprising a first frame member, a second frame member spaced axially apart from the first frame member, and an actuator extending through the first frame member and the second frame member, wherein the at least one actuator assembly comprises an outer sleeve having a tube portion abutting an outflow end portion of the second frame member and a first extension that extends distally from the tube portion and over the second frame member, a driver extending through the tube portion of the outer sleeve and engaging an end of the actuator, and a retaining element extending through aligned apertures in the outer sleeve and the second frame member such that the outer sleeve is retained against the outflow end portion of the second frame member; rotating the driver to cause corresponding rotation of the actuator to radially expand the prosthetic heart valve within the native heart valve while the outer sleeve is retained against the outflow end portion of the second frame member via the retaining element; releasing the retaining element from the second frame member and outer sleeve such that the outer sleeve and the second frame member are uncoupled from one another; and retracting the actuator assembly away from the prosthetic heart valve.

Example 51. The method of any example herein, particularly example 50, wherein releasing the retaining element from the second frame member and the outer sleeve includes applying a force to an end portion of the retaining element such that the retaining element flexes and is removed from the aligned apertures.

Example 52. The method of any example herein, particularly example 51, wherein the applied force is a pulling force that pulls the retaining element out of the aligned apertures, toward a central longitudinal axis of the frame of the prosthetic heart valve.

Example 53. The method of any example herein, particularly example 51 or example 52, wherein applying the force includes pulling a flexible member attached to the end portion of the retaining element and connected to a handle of the delivery apparatus proximally toward the handle via actuation of a control mechanism of the handle.

Example 54. The method of any example herein, particularly any one of examples 51-53, wherein the retaining element comprises two legs extending from the end portion of the retaining element and through the aligned apertures in the outer sleeve and the second frame member, the two legs engaging a first aperture of the aligned apertures disposed in the second frame member, and wherein releasing the retaining element includes pressing the two legs toward one another in response to the applied force to disengage the two legs from the first aperture.

Example 55. The method of any example herein, particularly any one of examples 51-53, wherein the outer sleeve further comprises a second extension extending from an opposite side of the outer sleeve than the first extension and over the second frame member, wherein the retaining element comprises two legs extending from the end portion of the retaining element and through the aligned apertures in the outer sleeve and the second frame member, each leg of the two legs further extending through and engaging a different one of two apertures in the second extension, and wherein releasing the retaining element includes pressing the two legs toward away from one another in response to the applied force to disengage the two legs from the two apertures in the second extension.

Example 56. The method of any example herein, particularly any one of examples 51-53, wherein the outer sleeve further comprises a second extension extending from an opposite side of the outer sleeve than the first extension and over the second frame member, the first and second extensions disposed on opposites sides of the second frame member, wherein each of the first extension and the second extension include two apertures therein that are spaced apart from one another, and wherein the retaining element comprises a flexible elongate retaining element that assumes a u-shape with a first leg extending through a first set of aligned apertures in the first and second extensions and the second frame member and a second leg extending through a second set of aligned apertures in the first and second extensions and the second frame member.

Example 57. The method of any example herein, particularly example 56, wherein releasing the retaining element includes pulling the flexible elongate retaining element through and away from the first set of aligned apertures and the second set of aligned apertures in response to the force applied to the end portion of the retaining element.

Example 58. The method of any example herein, particularly any one of examples 50-57, wherein retracting the actuator assembly away from the prosthetic heart valve includes actuating a control mechanism of a handle of the delivery apparatus that is connected to the outer sleeve to move the outer sleeve proximally toward the handle and away from the prosthetic heart valve.

Example 59. The method of any examples herein, particularly any one of examples 50-58, wherein the method is performed on a living animal or on a non-living simulation.

Example 60. A method comprising sterilizing the prosthetic heart valve, apparatus, and/or assembly of any example.

Example 61. A prosthetic heart valve of any one of examples 1-59, wherein the prosthetic heart valve is sterilized.

The features described herein with regard to any example can be combined with other features described in any one or more of the other examples, unless otherwise stated. For example, any one or more of the features of one actuator assembly can be combined with any one or more features of another actuator assembly. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus for a radially expandable prosthetic device, comprising:
an actuator assembly comprising:
an outer sleeve comprising a tube portion and an extension that extends distally from the tube portion, the extension comprising a first aperture in its distal end;
an actuation member extending through the tube portion of the outer sleeve; and
a retaining element configured to extend through the first aperture in the extension of the outer sleeve and hold the outer sleeve against an end portion of a frame of a prosthetic device, wherein the retaining element is biased into a first position where it holds the outer sleeve in engagement with the frame, and is configured to flex in response to a pulling force into a second position that enables the retaining element to be released from the first aperture, thereby enabling disengagement of the outer sleeve from the end portion of the frame of the prosthetic device.

2. The delivery apparatus of claim 1, wherein the retaining element comprises a head portion including a second aperture and two legs spaced apart from one another and extending from the head portion, wherein the two legs are configured to flex between the first position and the second position.

3. The delivery apparatus of claim 2, wherein the extension of the outer sleeve is a first extension, and wherein the outer sleeve comprises a second extension extending from an opposite side of the tube portion of the outer sleeve than the first extension, the first and second extensions spaced apart from one another.

4. The delivery apparatus of claim 3, wherein the first extension is longer than the second extension.

5. The delivery apparatus of claim 3, wherein the first extension and the second extension extend distally from the tube portion by a same amount, wherein the second extension comprises two second apertures that are spaced apart from one another, and wherein the first aperture of the first extension is configured to receive the two legs of the retaining element therethrough and each second aperture of the two second apertures of the second extension is configured to receive one of the two legs therethrough.

6. The delivery apparatus of claim 2, wherein each leg of the two legs comprises a bent end that is bent relative to a remainder of the leg, the bent end configured to further retain the retaining element within the first aperture and the outer sleeve in engagement with the frame.

7. The delivery apparatus of claim 2, wherein the head portion comprises a base from which the two legs extend, and wherein a width of the base is larger than a diameter of the first aperture such that the head portion remains exterior to the first aperture, on an outer surface of the extension.

8. The delivery apparatus of claim 1, wherein the extension of the outer sleeve is a first extension, wherein the outer sleeve comprises a second extension extending from an opposite side of the tube portion of the outer sleeve than the first extension, the first and second extensions spaced apart from one another, and wherein each of the first extension and the second extension include two apertures therein that are spaced apart from one another in a direction that is perpendicular to an axial direction of the actuator assembly.

9. The delivery apparatus of claim 8, wherein the retaining element is a flexible tubular retaining element, and wherein in the first position the retaining element assumes a U-shaped configuration with a first leg of the retaining element extending through a first aperture of the two apertures of the first extension and a first aperture of the two apertures of the second extension, the first apertures of the first and second extensions opposing one another across a gap separating the first and second extensions, and a second leg extending through a second aperture of the two apertures in the first extension and a second apertures in the two apertures in the second extension, the second apertures opposing one another across the gap separating the first and second extensions.

10. The delivery apparatus of claim 1, further comprising a handle, and wherein the actuator assembly extends distally from the handle.

11. The delivery apparatus of claim 10, further comprising a flexible element extending from the handle to an end portion of the retaining element and wherein the flexible element is configured to apply the pulling force to the end portion of the retaining element in response to actuation of a control mechanism of the handle.

12. An assembly comprising:
a prosthetic heart valve comprising:
a radially expandable and compressible frame, the frame comprising at least one actuation mechanism comprising:
a first frame member having a first inner bore and a second frame member having a second inner bore, the first and second frame members being spaced apart axially from one another; and
a threaded rod extending through the first and second inner bores; and
a delivery apparatus comprising:
a handle; and
at least one actuator assembly extending from the handle, the actuator assembly comprising:
an outer sleeve having a tube portion abutting an outflow end portion of the second frame member and a first extension that extends distally from the tube portion and over the second frame member;
a driver extending through the tube portion of the outer sleeve and engaging an end of the threaded rod, the driver configured to rotate the threaded rod to radially expand and/or compress the frame; and
a retaining element extending through a first aperture in the first extension of the outer sleeve and a second aperture in the second frame member, the retaining element configured to hold the outer sleeve in engagement with the second frame member, and wherein the retaining element is configured to flex and release from the first and second apertures in response to a pulling force applied to an end portion of the retaining element.

13. The assembly of claim 12, wherein the retaining element comprises a head portion and two legs spaced apart from one another extending from the head portion, wherein the legs are configured to flex under external force toward or away from one another, and wherein the first aperture and the second aperture are shaped to receive the legs therethrough.

14. The assembly of claim 13, wherein the head portion comprises a third aperture, and wherein the third aperture is attached to a flexible element that extends to a control mechanism of the handle that is configured to pull the flexible element proximally toward the handle and remove the retaining element from the outer sleeve and the second frame member.

15. The assembly of claim 13, wherein the first and second apertures are aligned with one another when the driver is coupled to the threaded rod, and wherein the second aperture extends through a thickness of the second frame member at a location offset from one side of the threaded rod.

16. The assembly of claim 15, wherein the legs are movable between a resting first position where the legs are pressed against the second aperture when the retaining element is coupled with the outer sleeve and the second frame member, and a flexed second position where the legs are pressed toward one another such that the legs can be released from the second aperture and the first aperture.

17. A method comprising:
advancing a distal end portion of a delivery apparatus toward a native heart valve, the delivery apparatus releasably coupled to a prosthetic heart valve by at least one actuator assembly, the prosthetic heart valve retained by the delivery apparatuses in a radially compressed state and comprising a frame comprising at least one actuation mechanism, the at least one actuation mechanism comprising a first frame member, a second frame member spaced axially apart from the first frame member, and an actuator extending through the first frame member and the second frame member, wherein the at least one actuator assembly comprises an outer sleeve having a tube portion abutting an outflow end portion of the second frame member and a first extension that extends distally from the tube portion and over the second frame member, a driver extending through the tube portion of the outer sleeve and engaging an end of the actuator, and a retaining element extending through aligned apertures in the outer sleeve and the second frame member such that the outer sleeve is retained against the outflow end portion of the second frame member;
rotating the driver to cause corresponding rotation of the actuator to radially expand the prosthetic heart valve within the native heart valve while the outer sleeve is retained against the outflow end portion of the second frame member via the retaining element;
releasing the retaining element from the second frame member and outer sleeve such that the outer sleeve and the second frame member are uncoupled from one another; and
retracting the actuator assembly away from the prosthetic heart valve.

18. The method of claim 17, wherein releasing the retaining element from the second frame member and the outer sleeve includes applying a force to an end portion of the retaining element such that the retaining element flexes and is removed from the aligned apertures.

19. The method of claim 18, wherein applying the force includes pulling a flexible member attached to the end portion of the retaining element and connected to a handle of the delivery apparatus proximally toward the handle via actuation of a control mechanism of the handle.

20. The method of claim 18, wherein the retaining element comprises two legs extending from the end portion of the retaining element and through the aligned apertures in the outer sleeve and the second frame member, the two legs engaging a first aperture of the aligned apertures disposed in the second frame member, and wherein releasing the retaining element includes pressing the two legs toward one another in response to the applied force to disengage the two legs from the first aperture.

21. The method of claim 18, wherein the outer sleeve further comprises a second extension extending from an opposite side of the outer sleeve than the first extension and over the second frame member, wherein the retaining element comprises two legs extending from the end portion of the retaining element and through the aligned apertures in the outer sleeve and the second frame member, each leg of the two legs further extending through and engaging a different one of two apertures in the second extension, and wherein releasing the retaining element includes pressing the two legs toward away from one another in response to the applied force to disengage the two legs from the two apertures in the second extension.

22. The method of claim 18, wherein the outer sleeve further comprises a second extension extending from an opposite side of the outer sleeve than the first extension and over the second frame member, the first and second extensions disposed on opposites sides of the second frame member, wherein each of the first extension and the second extension include two apertures therein that are spaced apart from one another, and wherein the retaining element comprises a flexible elongate retaining element that assumes a u-shape with a first leg extending through a first set of aligned apertures in the first and second extensions and the second frame member and a second leg extending through a second set of aligned apertures in the first and second extensions and the second frame member.
